(19) United States
(12) Patent Application Publication
(10) Pub. No.: US 2026/0083543 A1
(43) Pub. Date: Mar. 26, 2026

(54) A SURGICAL DEVICE

(71) Applicant: Marsal Innovation Pty Ltd., Canterbury (AU)

(72) Inventor: Salwan Hamoudi Musa AL-SALIHI, Canterbury (AU)

(21) Appl. No.: 19/126,579

(22) PCT Filed: Nov. 2, 2022
(86) PCT No.: PCT/AU2023/051105

§ 371 Date: May 1, 2025

(30) Foreign Application Priority Data
Nov. 2, 2022 (AU) 2022903269

(51) Int. Cl. A61F 2/00; A61B 17/42

(52) U.S. Cl. CPC A61F 2/0045; A61B 17/4241; A61B 2017/4225

(57) ABSTRACT

The present disclosure relates to a devices and methods for use in surgery. In particular, the present disclosure is directed to devices and methods for the treatment of pelvic organ prolapse conditions, and in particular for the treatment of uterine prolapse.

### CROSS-REFERENCE TO RELATED APPLICATIONS

The present application claims priority from Australian Provisional Patent Application No. 2022903269 filed on 2 Nov. 2022, the contents of which are incorporated herein by reference in their entirety.

### TECHNICAL FIELD

The present disclosure relates to a devices and methods for use in surgery. In particular, the present disclosure is directed to devices and methods for the treatment of pelvic organ prolapse conditions, and in particular for the treatment of uterine prolapse.

### BACKGROUND

Pelvic organ prolapse generally occurs when organs or other structures within the pelvic cavity descend from their normal anatomical positions. For example, uterine prolapse can occur when pelvic floor muscles and ligaments stretch and weaken, providing inadequate support for the uterus, leading to the uterus descending into and/or protruding from the vagina.

As such, pelvic organ prolapse typically occurs as a result of weakening or damage to the muscles and tissues that support the vagina, uterus and surrounding organs that otherwise function to hold them in place. Pelvic organ prolapse can affect women of any age, but is generally due to damage to supportive tissues during pregnancy and childbirth, the effects of gravity, a loss or lack of estrogen after menopause and/or repeated straining, all of which can weaken the pelvic floor.

Various surgical treatments for pelvic organ prolapse are available depending on the type and severity thereof. Most treatments involve utilizing implantable meshes to help provide support to or suspend prolapsed organs in the pelvic cavity (i.e., mesh hysteropexy). By way of example, mesh hysteropexy allows for uterine preservation by suspending the uterus to the sacral promontory. Such procedures, however, are not without their complications including mesh exposure and mesh injuries to surrounding organs. Additionally, in cases where chronic pain, complications or the need to have a hysterectomy are involved, the removal of implanted mesh can be difficult given its ability to interact and integrate with the surrounding pelvic tissues after placement.

Accordingly, there remains a need for improved surgical devices and methods for treating pelvic organ prolapse, such as uterine prolapse, in a patient in need thereof.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is broadly directed to a surgical device, and more particularly an implant, for assisting a surgeon during surgery, for the treatment of pelvic organ prolapse conditions, and in particular for the treatment of uterine prolapse such as hysteropexy surgery (i.e., surgical fixation of a displaced uterus), in a patient. The current disclosure is further directed to a surgical kit including said surgical device and methods of using the surgical device in hysteropexy surgery.

In a first aspect, the present disclosure provides a surgical device for performing hysteropexy in a subject, said device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject, wherein the first end section comprises an elongate opening disposed longitudinally in the elongate body and adapted to receive the second free end of the elongate body therethrough to facilitate formation of the closed loop.

Suitably, the first end section further comprises an expanded portion for contacting the outer surface of the cervix. In particular examples, the first end section comprises an expanded portion for contacting the outer surface of the cervix and a narrowed portion that comprises the elongate opening.

In a second aspect, the present disclosure relates to a surgical device for performing hysteropexy in a subject, said device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject, wherein the first end section comprises an expanded portion for contacting the outer surface of the cervix and a narrowed portion that comprises a fastening arrangement to facilitate formation of the closed loop.

In some examples, the fastening arrangement comprises an elongate opening disposed longitudinally in the elongate body and adapted to receive the second free end of the elongate body therethrough to facilitate formation of the closed loop.

For the device of the above aspects, the elongate body is suitably substantially flattened.

In particular examples of the above aspects, the elongate body is substantially formed of a silicone or a derivative thereof.

In certain examples of the above aspects, the fastening arrangement is configured to be releasable to facilitate reversible formation of the closed loop.

In various examples of the above aspects, the fastening arrangement is configured to facilitate adjustment of a size of the closed loop.

In some examples of the above aspects, the first end section and/or the expanded portion further comprise one or a plurality of apertures that facilitate securing the closed loop to a portion of the subject's cervix and/or uterus.

In particular examples of the above aspects, the elongate body comprises, consists of or consists essentially of a nonporous material. More particularly, the elongate body suitably comprises, consists of or consists essentially of a nonporous silicon material or a non-porous silicon-derivative material.

In certain examples of the above aspects, the second end section comprises one or a plurality of further openings disposed at or towards the second free end of the elongate band that facilitate securing the device to the anterior longitudinal ligament.

In various examples of the above aspects, the second end section comprises one or a plurality of removable segments disposed at or towards the second free end of the elongate band.

In a third aspect, the present disclosure provides a method of performing hysteropexy in a subject, said method including the steps of:
providing a surgical device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject, wherein the first end
   (a) an elongate opening disposed longitudinally in the elongate body and adapted to receive the second free end of the elongate body therethrough to facilitate formation of the closed loop; and/or
   (b) an expanded portion for contacting the outer surface of the cervix and a narrowed portion that comprises a fastening arrangement to facilitate formation of the closed loop;
positioning the first end section adjacent the cervix and/or uterus of the subject so as to extend at least partly therearound;
forming the closed loop of the first end section around the cervix and/or uterus of the subject; and
securing the second end section to the anterior longitudinal ligament of the subject.

Suitably, the present method further comprises the step of securing the first end section and/or the expanded portion to the cervix and/or uterus of the subject.

In some examples, the present method further comprises the step of making one or more incisions in a broad ligament of the subject to facilitate positioning of the first end section.

Suitably, the surgical device is that of the first or second aspects.

In a fourth aspect, the present disclosure provides a kit comprising the surgical device of the first or second aspects and optionally instructions for use.

Suitably, the kit is for use in the method of the third aspect.

Any embodiment herein shall be taken to apply mutatis mutandis to any other embodiment unless specifically stated otherwise.

It will be appreciated that the indefinite articles “a” and “an” are not to be read as singular indefinite articles or as otherwise excluding more than one or more than a single subject to which the indefinite article refers.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

In order that the present invention may be readily understood and put into practical effect, reference will now be made to the accompanying illustrations, wherein like reference numerals are used to refer to like elements.

FIG. 1: is a top view (A) and a side view (B) of an embodiment of a surgical device for hysteropexy surgery.

FIG. 2: is a side view of the surgical device of FIG. 1 implanted in the pelvic cavity of a subject.

FIG. 3: is an anterior view of the surgical device of FIG. 1 implanted in the subject's pelvic cavity.

### DETAILED DESCRIPTION

The present disclosure relates to a surgical device or implant for use during hysteropexy surgery that may facilitate relatively quick and easy positioning and fixation within the pelvic cavity, whilst minimising the risk of post-surgical complications, such as erosion, exposure and trauma to the underlying cervical and/or uterine tissue. A previous example of a hysteropexy device in provided in PCT/AU2022/050399, which is incorporated in its entirety by reference herein.

While the principles described herein are based on methods of providing surgical devices for humans, and more particularly female humans, this invention may also be extended to other mammals such as livestock (e.g. cattle, sheep), performance animals (e.g. racehorses) and domestic pets (e.g. dogs, cats), although without limitation thereto.

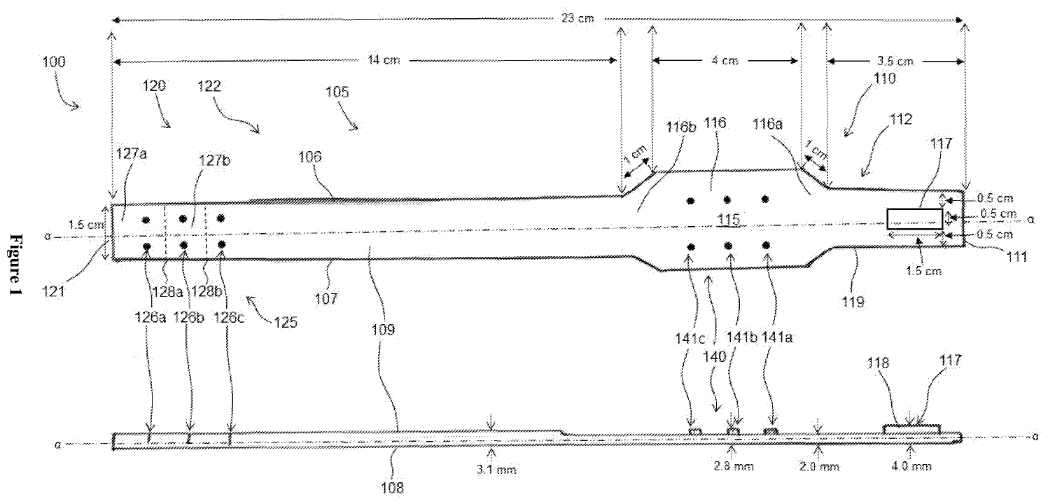

A first embodiment of an implantable surgical device 100 is provided in FIG. 1. The surgical device 100 includes an integrally formed flexible and elongate band or body 105 that is substantially flattened and defines a long or longitudinal axis a, first and second free ends 111,121, first and second lateral edges 106,107 and inner and outer surfaces 108,109 thereof. The elongate body 105 defines a proximally positioned first end section 110 and a distally positioned second end section 120. The first end section 110 and the second end section 120 together define the elongate band 105 as being substantially straight therebetween. Suitably, the first and second lateral edges 106,107 of the elongate band 105 are rounded so as to minimize the potential of compromising underlying cervical and uterine tissue.

As can be seen in FIG. 1, the first end section 110 includes a flared or expanded portion 115 disposed at or towards a distal end thereof and adjacent a proximal end of the second end section 120. It is envisaged, however, that the expanded portion 115 may disposed at any position along the first end section 110, such as approximately centrally therein or close to or adjacent a proximal end (i.e., the first free end 111) thereof. Additionally, the first end section 110 includes a narrowed portion 119 that extends proximally from the expanded portion 115 to the first free end 111 thereof. Just proximal of a middle section or mid-point of the elongate band 105, the expanded portion 115 extends radially outwards a similar distance from each of the respective first and second lateral edges 106, 107 to define a contact surface 116 that is of wider dimensions than the remainder of the elongate band 105 and is further defined by respective tapered portions 116a, b at either end thereof. Conversely, the narrowed portion 119 has a width that is of similar or substantially the same dimensions as that of the second end section 120.

The expanded portion 115 advantageously provides a greater contact or attachment surface area to the cervix 10, thereby providing greater stability to the device 100 under traction in the pelvic cavity 40. Additionally, it may prevent or minimise surface damage or reactions in the cervix 10 by providing a greater surface area across which to spread or distribute traction forces exerted on the anterior portion of the cervix 10.

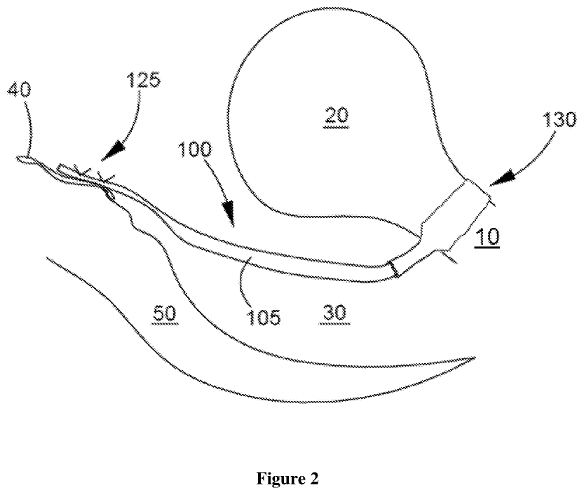

The inner surface 108 of the contact surface 116 of the expanded portion 115 is of suitable dimensions (e.g., width and length) so as to be configured to wrap around and contact an outer surface of a subject's cervix 10 and/or uterus 20, as shown in FIG. 2. By virtue of this arrangement, the expanded portion 115 is designed to encompass and contact at least a portion, such as anterior, lateral and/or posterior portions, of an outer surface of a subject's cervix 10 and optionally an adjacent lower section of their uterus 20.

In the embodiment provided in FIG. 1, the expanded portion 115 comprises a non-porous material, as described herein. Alternatively, it is contemplated that the expanded portion 115 may include a porous or fenestrated portion (not shown) comprising a plurality of openings and configured to allow passage therethrough of a fluid or exudate from the adjacent surface of the cervix 10 and/or uterus 20. In such alternative examples, it is contemplated that the remainder of the surgical device 100 comprises, consists of or consists essentially of a non-porous material, such as a non-porous silicon material.

The expanded portion 115 of the surgical device 100 also includes a securing arrangement 140 for securing a closed loop 130 to the cervix 10 and/or uterus 20. The securing arrangement 140 includes three pairs of serially and evenly spaced apart openings or eyelets 141a-c positioned approximately centrally along the expanded portion 115 of the first end section 110. The openings 141a-c are of suitable dimensions (e.g., 0.5, 0.75, 1.0, 1.25, 1.5, 2.0, 2.25, 2.5 mm or any range therein) for receiving one or more sutures, staples or other surgical fasteners therethrough and thereby facilitate securing the closed loop 130 as defined by the expanded portion 115 to a surface of the subject's cervix 10 and/or uterus 20. In the embodiment provided, the openings 141a-c include a thickened reinforcing portion or ridge disposed therearound so as to inhibit or prevent ripping of these structures when under tension. Whilst FIG. 1 demonstrates three pairs of the openings 141a-c in the expanded portion 115, it is envisaged that any number (e.g., 1, 2, 3, 4, 5, 6 etc) and arrangement (e.g., single, pairs or triplets) of the openings 141a-c may be utilized for the securing arrangement 140.

Alternative embodiments of the securing arrangement 140 for securing the expanded portion 115 and the closed loop 130 formed therefrom to the cervix 10 and/or uterus 20 are envisaged. For instance, the securing arrangement 140 may instead include one or more spaced apart loops or eyelets (not shown) positioned along the first lateral edge 106 of the expanded portion 115 of the elongate band 105 and extending outwardly therefrom. The eyelets can be of suitable dimensions for receiving one or more sutures, staples or other surgical fasteners therethrough and thereby facilitate securing the closed loop 130 to a surface (e.g., an anterior, lateral and/or posterior surface) of the subject's cervix 10 and/or uterus 20.

In order to minimize or prevent slipping or displacement of the device 100 described herein off the subject's cervix 10 and onto, for example, the body of the uterus 20, the expanded portion 115 may, in addition or alternatively to the openings 141a-c, comprise a textured surface or portion, such as a high friction or patterned surface (not shown), at least partly along an inner surface of the closed loop 130. The textured surface is designed for contacting or abutting a surface of the subject's cervix 10 and/or uterus 20, particularly anterior aspects thereof, to inhibit movement of the closed loop 130 relative thereto.

As can be observed in FIG. 1, an elongate opening or slit 117 is disposed at or towards a proximal end 112 of the first end section 110 of the elongate band 105 in the narrowed portion 119 thereof. A long axis of the elongate slit 117 is disposed longitudinally so as to extend axially along or parallel to a long or longitudinal axis a of the elongate band 105, rather than transversely thereto. This arrangement is designed to produce an appropriate level of twisting or torsion of the elongate body 105 once the surgical device 100 is appropriately positioned within the pelvic cavity 40. More particularly, this longitudinal arrangement of the slit 117 relative to the longitudinal axis of the elongate band 105 advantageously functions to reduce the twisting in the second end section 120 of the device 100 and thereby maximizes the stability of the device 100 when in position and under traction in the pelvic cavity 40. At the same time, this arrangement may reduce the structural stress on the elongate band 105 at the point where it twists and thereby minimizes the likelihood of failure or breaking of the device 100.

In alternative embodiments, it is contemplated that the expanded portion 115 may extend proximally to, and thereby encompass, the first free end 111 of the surgical device 100 (such as illustrated in FIG. 10 of PCT/AU2022/050399) so as to include the elongate slit 117 therein. For such embodiments, the first end section 110 suitably does not include a narrowed portion 119.

The elongate slit 117 is bordered or surrounded by a thickened reinforcing portion 118 that is disposed around the respective opening thereof and extends outwardly from the outer surface 109. This arrangement is designed to inhibit or prevent ripping of the elongate slit 117 when under tension in the pelvic cavity 40. From FIG. 1, the thickened reinforcing portion 118 is surrounded or bordered on each side by relatively thinner portions of the first end section 110, which allows for easier foldability and provides a leading edge at the first free end 111 for smoother insertion through surgical incisions, such as those made in the broad ligament. In alternative embodiments, the first end section 110 may include a thickened portion that extends distally from the first free end 111 to just beyond the elongate slit 117 so as to again provide reinforcement thereto.

As illustrated in FIG. 1B, the first end section 110 is of thinner dimensions or narrower in cross-section than the second end section 120. In this regard, the boundary between the first and second end sections 110,120 is defined by a change in thickness or narrowing therebetween and/or the tapered portion 116b at the distal end of the expanded portion 115 of the elongate band 105. The relatively increased thickness of the second end section 110 along its length thereof is intended to reduce or minimize stretching of the surgical device 100 under relative high force loads once suitably implanted within the pelvic cavity 40.

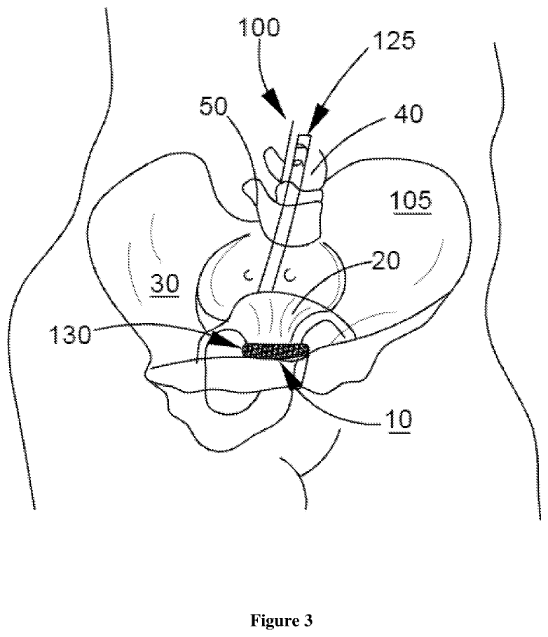

The elongate slit 117 is of suitable dimensions for receiving the second free end 121 and the second end section 120 of the elongate band 105 therethrough. In the embodiment provided herein, the elongate slit 117 has an internal length of about 1.5 cm and an internal width of about 0.5 cm, but other suitable dimensions are contemplated. By virtue of this cooperating fastening arrangement between the elongate slit 117 and the second end section 120, the expanded portion 115 is reversibly or releasably operable between open and closed positions so as to be capable of forming the closed loop 130 around the cervix 10 and/or uterus 20 when appropriately positioned in the pelvic cavity 30, as shown in FIGS. 2 and 3. In the event where post-surgical complications occur or the subject subsequently requires a hysterectomy, this arrangement also allows for a relatively easier removal of the device 100 compared to that for traditional mesh-based implants. To this end, there is little or no tissue interaction between the elongate band 105 and the surrounding tissue compared to that for mesh-based implants (e.g., no collagen deposition and no in-growth of tissue around the device 100 unlike mesh-based implants of the prior art).

This fastening arrangement also allows for the closed loop 130 to naturally tighten around the cervix 10 over time. Alternatively or additionally, the closed loop 130 may be adjusted manually by a surgeon to a size appropriate for the subject's cervix 10 and its positioning within the pelvic cavity 30.

Additionally, the skilled person will appreciate that alternative fastening arrangements as are known in the art may also be used for the device 100 described herein, such as a button snap arrangement, a lasso-like arrangement, a buckle clasp arrangement and loop fasteners. Additionally, the fastening arrangement suitably provides different or alternative loop forming engagement or securement locations, which are selectable by the surgeon depending upon the anatomical size of the subject's cervix and/or uterus or other anatomical or surgical considerations.

Referring to FIGS. 2 and 3, a distally positioned securable portion 125 of the second end section 120 of the elongate band 105 is adapted to be secured to the anterior longitudinal ligament (ALL) 40, which runs along the anterior surface of the vertebral bodies and intervertebral discs of the spine 50. As illustrated in FIG. 1, the securable portion 125 includes three pairs of adjacent suture openings, apertures or slits 126a-c serially arranged along the long axis of the elongate band 105. Each pair of suture openings 126a-c is dimensioned to receive one or more sutures (or other fastening element known in the art) therethrough to connect the securable portion 125 to the ALL 40.

The device 100 is also configured to allow for altering a length of the second end section 120. To this end, the securable portion 125 further includes two removable segments 127a-b positioned serially from the second free end 121 of the elongate band 105. The two removable segments 127a-b are further defined or delineated by serially and equidistant positioned indented or molded indicators or guides 128a-b that extend transversely around the inner and outer surfaces 108,109 and the first and second lateral edges 106,107 of the elongate band 105 so as to function as trim guides for a user. To this end, the guides 128a-b are positioned approximately half-way between slits 126a and 126b and slits 126b and 126c respectively, such that the pair of adjacent slits 126a are disposed within the removable segment 127a and the pair of adjacent slits 126b are positioned within the removable segment 127b. By virtue of this arrangement, a surgeon is able to remove or trim one or more removable segments 127a-b from the distal end 122 of the elongate band 105 to alter or adjust a length of the securable portion 125 as required by, for example, the anatomical size of the subject in question and/or the degree of displacement of the subject's cervix 10 and uterus 20, such that these organs can be appropriately suspended or positioned in the pelvic cavity 30 upon placement of the device 100.

It will be appreciated that the surgical devices described herein may include any number (e.g., 1, 2, 3, 4, 5, 6, 7, 8 etc) of the pair of suture openings 126a-c and/or the removable segments 127a-b so as to facilitate suitable securement of the device 100 to the ALL 40. Additionally, the removable segments 127a-b can be, for example, about 0.5 cm to about 5 cm long (e.g., about 0.5, 0.75, 1.0, 1.25, 1.5, 1.75, 2.0, 2.25, 2.5, 2.75, 3.0, 3.25, 3.5, 3.75, 4.0, 4.25, 4.5, 4.75 or 5.0 cm, inclusive of any range therein). In the example in FIG. 1, each of the removable segments 127a-b is about 1.0 cm in length. Furthermore, each of the openings of the suture openings is at least about 0.5 mm, at least about 1.0 mm, at least about 1.5 mm or at least about 2.0 mm (e.g., about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 mm or any range therein) in diameter.

As used herein, the terms “approximately” and “about” refer to tolerances or variances associated with numerical values recited herein (e.g., the numerical value ±0.5%, 1%, 5% or 10%). The extent of such tolerances and variances are well understood by persons skilled in the art. Typically, such tolerances and variances do not compromise the structure, function and/or implementation of the devices and methods described herein.

As illustrated in FIG. 1, the adjacent suture openings 126a-c have a diameter of about 2.0 mm so as to cater for an appropriately size suture hook or needle, although any suitable diameter is envisaged (e.g., about 1.0 mm, 1.5 mm, 2.0 mm, 2.5 mm, 3.0 mm, 3.5 mm, 4.0 mm or any range therein). Additionally, and although not demonstrated in FIG. 1, the adjacent suture openings 126a-c may include a thickened reinforcing portion disposed at least partly around the respective openings thereof to inhibit or prevent ripping of these structures when under tension.

Suitably, the surgical device 100 described herein is integrally constructed of a medical-grade or implant-grade silicone, a derivative thereof or other suitable medical-grade or implant-grade polymer or flexible non-resorbable material (e.g., polypropylene, polyester, polyethylene, urethane, polyurethane, copolymers, and block copolymers) as are known in the art. In some embodiments, the surgical devices described herein include a reinforcing mesh, such as a fine titanium mesh, disposed or contained within the medical grade silicone or other suitable medical material that substantially makes up the device. The reinforcing mesh suitably improves the tensile strength of the surgical device, whilst also allowing for flexibility thereof. In other embodiments, the surgical device may comprise a material that is substantially transparent or translucent so as to allow for the visualization of underlying anatomic structures.

In certain embodiments, the surgical device 100 described herein is constructed or substantially constructed of a nonporous material, such as a nonporous medical-grade or implant-grade silicone or derivative thereof. The term “nonporous” means that, aside from the openings for receiving sutures, the surgical device (or a substrate thereof) is substantially free of pores. To this end, the surgical device of the invention suitably does not comprise or is not made of a surgical or implantable mesh, mesh implant or mesh material (e.g., a synthetic mesh, such as a polypropylene mesh).

In particular embodiments, the elongate band described herein has a width, such as an average width, of about 0.5 cm to about 5.0 cm (e.g., about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 cm or any range therein) between respective edges thereof. As noted above, the first and second end sections and the expanded portion of the elongate band may be of different widths. Suitably, the expanded portion and optionally the first end section is wider than the second end section to increase the contact surface area of the closed loop for the subject's cervix. In certain embodiments, the expanded portion has a width, such as an average width, inclusive or exclusive of any tapered portions thereof, of about 1.0 cm to about 4.0 cm (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 cm or any range therein), more particularly about 1.5 cm to about 3.5 cm, even more particularly about 2.0 cm to about 3.0 cm or yet even more particularly about 2.5 cm. In some embodiments, the second end section has a width of about 1.0 cm to about 2.0 cm (e.g., about 1.0, 1.1, 1.2, 1.3, 1.4, 1.5, 1.6, 1.7, 1.8, 1.9, 2.0 cm or any range therein), or more particularly about 1.5 cm. In particular embodiments, the first end section outside of or separate from the expanded portion has a width of about 1.0 cm to about 2.0 cm (e.g., about 1.0, 1.1, 1.2, 1.3, 1.4, 1.5, 1.6, 1.7, 1.8, 1.9, 2.0 cm or any range therein), or more particularly about 1.5 cm.

Suitably, the elongate band described herein has a length of about 20 cm to about 40 cm (e.g., about 20, 21, 22, 23, 24, 25, 26, 27, 28, 29, 30, 31, 32, 33, 34, 35, 36, 37, 38, 39, 40 cm or any range therein). In particular embodiments, the elongate band described herein has a length of about 20 cm to about 30 cm. In various embodiments, the elongate band described herein has a length of about 23 cm. In some embodiments, the elongate band described herein has a length of about 25 cm. In other embodiments, the elongate band described herein has a length of about 28 cm.

In particular embodiments, the first end section described herein, inclusive of the expanded portion, is about 7.5 cm to about 15.0 cm in length (e.g., about 7.5, 8.0, 8.5, 9.0, 9.5, 10.0, 10.5, 11.0, 11.5, 12.0, 12.5, 13.0, 13.5, 14.5, 15.0 cm in length or any range therein). In some embodiments, the first end section is about 9 cm in length. In various embodiments, the first end section is about 10 cm in length. In other embodiments, the first end section is about 13 cm in length.

In some embodiments, the expanded portion is about 4.0 cm to about 15 cm (e.g., about 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0 cm in length or any range therein) or more particularly about 4.0 cm to about 10 cm (e.g., about 4.0, 4.5, 5.0, 5.5, 6.0, 6.5, 7.0, 7.5, 8.0, 8.5, 9.0, 9.5, 10.0 cm in length or any range therein). In various embodiments, the expanded portion is about 10 cm in length. In other embodiments, the expanded portion is about 9 cm in length. In other embodiments, the expanded portion is about 7 cm in length. In particular embodiments, the expanded portion is about 6 cm in length.

In various embodiments, the second end section described herein is about 10 cm to about 20 cm in length (e.g., about 10.0, 10.5, 11.0, 11.5, 12.0, 12.5, 13.0, 13.5, 14.5, 15.0, 15.5, 16.0, 16.5, 17.0, 17.5, 18.0, 18.5, 19.0, 19.5, 20.0 cm in length or any range therein) or more particularly about 12 cm to about 18 cm in length. In particular embodiments, the second end section is about 14 cm in length. In other embodiments, the second end section is about 15 cm in length. In certain embodiments, the first end section is about 9 cm in length and the second end section is about 14 cm in length. In other embodiments, the first end section is about 10 cm in length and the second end section is about 15 cm in length.

Further, the elongate band described herein is preferably relatively thin, whilst maintaining suitable tensile strength, similar to that of a mesh for use in hysteropexy. In some embodiments, the elongate band described herein has a thickness, or more particularly a cross-sectional thickness, of about 1.0 mm to about 5.0 mm (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 mm or any range therein), more particularly about 2.0 mm to about 4.0 mm or even more particularly about 2.5 mm to about 3.5 mm. In particular embodiments, the elongate band described herein has a thickness, such as an average thickness, of about 3.0 mm.

As noted above, the elongate band may have one or more portions of a different thickness along its length. Suitably, the first end section is thinner in cross-section than the second end section to allow for improved flexibility when positioned around the subject's cervix within the pelvic cavity. In particular embodiments, the first end section has a thickness (e.g., an average thickness) of about 1.0 mm to about 3.0 mm (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 mm or any range therein), and the second end section has a thickness of about 2.5 mm to about 4.5 mm (e.g., about 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5 mm or any range therein). In particular embodiments, the first end section has a thickness of about 1.5 mm. In other embodiments, the first end section has a thickness of about 2.0 mm. In certain embodiments, the second end section has a thickness of about 3.0 mm. In various embodiments, the second end section has a thickness of about 4.0 mm. In some embodiments, the first end section has a thickness of about 2.0 mm and the second end section has a thickness of about 3.0 mm.

For illustration purposes, the following details with respect to methods of surgical placement relate to the surgical device 100. Moreover, embodiments of the below hysteropexy method may be performed using any method known in the art, such as robotically, laparoscopically, or in open surgery through a laparotomy incision.

Prior to implantation of the surgical device 100 hereinbefore described, the patient's pelvic cavity is exposed and the uterus 20 and cervix 10 are visualised. The uterus 20 is then pulled dorsally and apically so that the anterior aspect of the cervix 10 is exposed. Small incisions are then made with the use of scissors and/or cautery in the broad ligament on either side thereof and adjacent the cervix 10. Advantageously, the incisions to be made in the broad ligament are smaller and more discrete than those required for mesh hysteropexy. The expanded portion 115 of the surgical device 100 can then be placed in contact with the anterior aspect of the cervix 10 (and optionally the uterus 20) with the proximal end 112 of the elongate band 105 being fed through one incision in the broad ligament and the distal end 122 of the elongate band 105 being fed through the other incision in the broad ligament.

The second free end 121 of the second end section 120 can then be fed or drawn through the elongate slit 117 at the proximal end 112 of the elongate band 105 so as to form a suitably dimensioned closed loop 130 surrounding the subject's cervix 10, as shown in FIGS. 2 and 3. As noted above, the size of the closed loop 130 may be altered or adjusted as determined by the surgeon. In this regard, the join of the closed loop 130 is to be positioned in the midline of the pelvic cavity 40 and adjacent a posterior aspect of the cervix 10. The closed loop 130 or the expanded portion 115 may then be attached to the cervix 10, such as an anterior aspect thereof, by way of the securing arrangement 140 and sutures or the like.

The cervix 10 and uterus 20 are then lifted or suspended to an appropriate position within the pelvic cavity 30. Optionally, one or more removable segments 127a-b may then be trimmed from the securable portion 125 so as to achieve an appropriate length of the elongate band 105 that maintains the cervix 10 in this suspended position. The securable portion 125 of the elongate band 105 is then attached to the ALL 40 at the sacral promontory of the sacral spine 50 on to the anterior longitudinal ligament by way of permanent sutures (like Gore-Tex or prolene) or other suitable fastening element. Any remaining incisions are surgically closed and the device may be covered with peritoneum as per surgical steps relevant to the procedure. It is also envisaged that additional surgical procedures, such as oophorectomy and/or burch colposuspension may be executed at the time of the aforementioned hysteropexy surgery.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

1. A surgical device for performing hysteropexy in a subject, said device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject, wherein the first end section comprises an elongate opening disposed longitudinally in the elongate body and adapted to receive the second free end of the elongate body therethrough to facilitate formation of the closed loop.

2. The surgical device of claim 1, wherein the first end section comprises an expanded portion for contacting the outer surface of the cervix and a narrowed portion that comprises the elongate opening.

3. A surgical device for performing hysteropexy in a subject, said device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject, wherein the first end section comprises an expanded portion for contacting the outer surface of the cervix and a narrowed portion that comprises a fastening arrangement to facilitate formation of the closed loop.

4. The surgical device of claim 3, wherein the fastening arrangement comprises an elongate opening disposed longitudinally in the elongate body and adapted to receive the second free end of the elongate body therethrough to facilitate formation of the closed loop.

5. The device of claim 1, wherein the elongate body is substantially flattened.

6. The device of claim 1, wherein the elongate body is substantially formed of a silicone or a derivative thereof.

7. The device of claim 1, wherein the fastening arrangement is configured to be releasable to facilitate reversible formation of the closed loop.

8. The device of claim 1, wherein the fastening arrangement is configured to facilitate adjustment of a size of the closed loop.

9. The device of claim 1, wherein the first end section and/or the expanded portion further comprise one or a plurality of apertures that facilitate securing the closed loop to a portion of the subject's cervix and/or uterus.

10. The device of claim 1, wherein the elongate body comprises, consists of or consists essentially of a nonporous material.

11. The device of claim 1, wherein the second end section comprises one or a plurality of further openings disposed at or towards the second free end of the elongate band that facilitate securing the device to the anterior longitudinal ligament.

12. The device of claim 1, wherein the second end section comprises one or a plurality of removable segments disposed at or towards the second free end of the elongate band.

13. A method of performing hysteropexy in a subject, said method including the steps of:
providing a surgical device comprising a flexible elongate body defining first and second free ends and having a first end section that is configured to be formed into a closed loop for extending at least partly around an outer surface of the cervix of the subject, and a second end section extending from the closed loop that is configured to be secured to the anterior longitudinal ligament of the subject, wherein the first end section comprises:
(a) an elongate opening disposed longitudinally in the elongate body and adapted to receive the second free end of the elongate body therethrough to facilitate formation of the closed loop; and/or
(b) an expanded portion for contacting the outer surface of the cervix and a narrowed portion that comprises a fastening arrangement to facilitate formation of the closed loop;
positioning the first end section adjacent the cervix and/or uterus of the subject so as to extend at least partly therearound;
forming the closed loop of the first end section around the cervix and/or uterus of the subject; and
securing the second end section to the anterior longitudinal ligament of the subject.

14. The method of claim 13, further comprising the step of securing the first end section and/or the expanded portion to the cervix and/or uterus of the subject.

15. The method of claim 13, further comprising the step of making one or more incisions in a broad ligament of the subject to facilitate positioning of the first end section.

16. The method of claim 13, wherein the surgical device is that of claim 1.

17. (canceled)

18. (canceled)

19. The method of claim 13, wherein the surgical device is that of claim 3.

20. The device of claim 3, wherein the elongate body is substantially flattened.

21. The device of claim 3, wherein the elongate body is substantially formed of a silicone or a derivative thereof.

22. The device of claim 3, wherein the fastening arrangement is configured to be releasable to facilitate reversible formation of the closed loop.
